(19) United States
(12) Patent Application Publication
(10) Pub. No.: US 2006/0200072 A1
(43) Pub. Date: Sep. 7, 2006

(54) Needleless access port valves

(76) Inventor: Peter W. Peppel, Nazareth, PA (US)

Correspondence Address: CHRISTIE, PARKER & HALE, LLP, PO BOX 7068, PASADENA, CA, 91109-7068, US

(21) Appl. No.: 11/070,486

(22) Filed: Mar. 2, 2005

(51) Int. Cl. A61M 31/00

(52) U.S. Cl. 604/93.01; 604/523; 604/247; 604/905; 251/149.1

(57) ABSTRACT

Needleless access port valves are generally discussed herein with particular discussions extended to needleless access port valves comprising a self-closing slip port. The slip port is configured to receive a medical implement and has a first flattened configuration before insertion of the medical implement and a second less flattened configuration after the medical implement is inserted therein and received thereby. The slip port is self-closing subsequent to removing the medical implement. The slip port of the type described may be incorporated in a Y-site or an injection site.

Needleless access port valves are generally discussed herein with particular discussions extended to needleless access port valves comprising a self-closing slip port.

### BACKGROUND

Needleless access port valves are widely used in the medical industry for accessing an IV line and/or the internals of a patient or subject. Generally speaking, prior art valves utilize a valve housing in combination with a moveable internal plug or piston to control the flow of fluid through a valve. The plug or piston may be moved by a syringe or a medical implement to open the inlet of the valve for accessing the interior cavity of the valve. When a fluid is delivered through the valve, fluid flow typically flows around the outside of the plug or piston in the direction towards the outlet. Upon removal of the syringe or medical implement, the plug or piston returns to its original position, either un-aided or aided by a biasing means, such as a spring or a diaphragm.

In some prior art valves, when the syringe or medical implement pushes the plug or piston, the plug or piston is pierced by a piercing device, such as a spike. The spike typically incorporates one or more fluid channels for fluid flow flowing through the pierced piston and then through the fluid channels in the spike. In yet other prior art valves, a self-flushing or positive flush feature is incorporated to push residual fluids confined inside the interior cavity of the valve to flow out the outlet when the syringe or medical implement is removed.

While prior art needleless access port valves are viable options for their intended applications, there remains a need for alternative needleless access port valves.

### SUMMARY

The present invention may be implemented by providing a needleless injection port valve comprising a valve body, a first port comprising an exterior surface and an interior surface defining a lumen, and a second port; the first port comprising a first configuration in which a first section of the interior surface contacts a second section of the interior surface, and a first section of the exterior surface adjacent the first section of the interior surface is spaced apart from a second section of the exterior surface adjacent the second section of the interior surface by a first distance; and the first port comprising a second configuration in which the first and second sections of the interior surface are caused to be spaced apart from one another by at least one of a medical implement and a force acting on the exterior surface of the first port, and the first and second sections of the exterior surface are spaced apart from one another by a second distance, which is larger than the first distance.

The present invention may also be practiced by providing a needleless injection port valve comprising a valve body, a first port comprising an exterior surface and an interior surface defining a lumen, and a second port; wherein the lumen has a shape of a funnel having a first length measured along a first plane and a second length measured along a second plane; and wherein the first length decreases and the second length increases when the first port moves from a closed position to an open position.

In yet other aspects of the present invention, there is provided a needleless injection port valve comprising a valve body, a first port comprising an exterior surface and an interior surface defining a lumen, and a second port; wherein at least two threaded sections are positioned on the exterior surface of the first port, and wherein the two threaded sections move away from one another when the first port moves from a closed position to an open position.

Other aspects and variations of the valve assemblies summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is a semi-schematic front view of a needleless injection port valve provided in accordance with aspects of the present invention;

FIG. 2 is a semi-schematic side view of the valve of FIG. 1;

FIG. 3 is a semi-schematic cross-sectional side view of the valve of FIG. 2 taken along the same viewing plane;

FIG. 4 is a semi-schematic front view of an alternative needleless injection port valve provided in accordance with aspects of the present invention;

FIG. 5 is a semi-schematic side view of the valve of FIG. 4;

FIG. 6 is a semi-schematic cross-sectional side view of the valve of FIG. 5 taken along the same viewing plane;

FIG. 7 is a semi-schematic cross-sectional view of the valve of FIG. 4 taken along the same viewing plane;

FIG. 8 is a semi-schematic front view of yet another alternative needleless injection port valve provided in accordance with aspects of the present invention;

FIG. 9 is a semi-schematic side view of the valve of FIG. 8;

FIG. 10 is a semi-schematic cross-sectional side view of the valve of FIG. 9 taken from the same viewing plane; and

FIG. 11 is a semi-schematic cross-sectional view of the valve of FIG. 8 taken from the same viewing plane.

### DETAILED DESCRIPTION

The detailed description set forth below in connection with the appended drawings is intended as a description of the presently preferred embodiments of needleless access port valves or backcheck valves (herein “valves”) provided in accordance with aspects of the present invention and is not intended to represent the only forms in which the present invention may be constructed or utilized. The description sets forth the features and the steps for constructing and using the valves of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

Referring now to FIG. 1, a semi-schematic partial perspective view of an exemplary needleless injection port valve provided in accordance with aspects of the present invention is shown, which is generally designated 10. In one exemplary embodiment, the needleless injection port valve 10, herein Y-site valve or “valve 10”, comprises a valve body 12 comprising a first port 14, a second port 16, and a third port 18, which may sometime be referred to as an inlet port or a slip port. As is well known in the art, the first port 14 and the second port 16 are in constant fluid communication with one another while the slip port 18 is only in fluid communication with the two ports 14, 16 when opened by a medical implement, as further discussed below. Note that the name designation for the three ports (i.e., first, second, and third) can vary. In other words, a first port can alternatively be labeled a second port.

In one exemplary embodiment, the first and second ports 14, 16 are tubing ports adapted to receive tubing from an IV set or the like. Fluid flow through the tubing (not shown) is typically in the direction of the first port 14 towards the second port 16. The slip port 18 is a port for either supplementing or introducing fluid, via a medical implement, such as a syringe, to blend or commingle with fluid flow from between two ports. Alternatively, a fluid or blood sample can be withdrawn from the slip port. In one exemplary embodiment, the slip port 18 comprises a self-closing opening 20 formed from a resilient material. In a preferred embodiment, the valve 10, and hence the slip port 18, is integrally formed from a thermoplastic elastomer (TPE). In one exemplary embodiment, the TPE is a member of the copolyamide (COPA) family of thermoplastic elastomers. In a preferred embodiment, the COPA is copolyamide thermoplastic elastomer having a commercial trade name PEBAX®. However, other TPEs may also be used to make the valve housing 12, including thermoplastic polyurethanes (TPUs), styrenic thermoplastic elastomers, thermoplastic polyolefins (TPOs), copolyesters (COPEs), and thermoplastic vulcanizate elastomeric alloys (TPVs). Optionally, the TPEs may be cross-linked either chemically or by irradiation to alter their characteristics. In one exemplary embodiment, one or more colors are incorporated in the material. Preferably, the material has a translucent pantone green tone. Alternatively, an opaque material with one or more color tones may also be incorporated.

In one exemplary embodiment, the opening 20 has a shape of a slit formed by either cutting or molding the slit. A fluid tight seal is then provided by shrinking the slip port 18 or by post mold mechanical setting to set the slit. The final slip port configuration resembles a flattened cylindrical port in which the opening 20 comprises a generally linear slit located at an upper port section 21 that gradually tapers outwardly into a base port section 23. Luer threads 22 are optionally incorporated for engaging corresponding threads on a male luer connector (not shown). When penetrated by a medical implement (not shown), the opening 20, and hence the port 18 at the upper port section 21, expands to accommodate the male tip of a medical implement. As the opening 20 expands, the threads 22 project outwardly to engage corresponding threads on the threaded collar of the medical implement. In the closed configuration shown, the inlet 20 has a smooth contour for easy swabbing or cleaning, if necessary. The inlet is resilient and self-closes when not caused to be opened by externally applied forces or by a medical implement.

In one exemplary embodiment, levers 24 for squeezing are incorporated. The levers 24 are preferably integrally formed with the valve body 12. The levers 24 each comprises an outer actuating pad 26 connected to the valve body via a rib section 28. A pair of applied forces on the two actuating layers 26 in opposite directions will open the slit 20 and permit the male tip of a medical implement to penetrate the lumen defined by the slip port 18, as further discussed below.

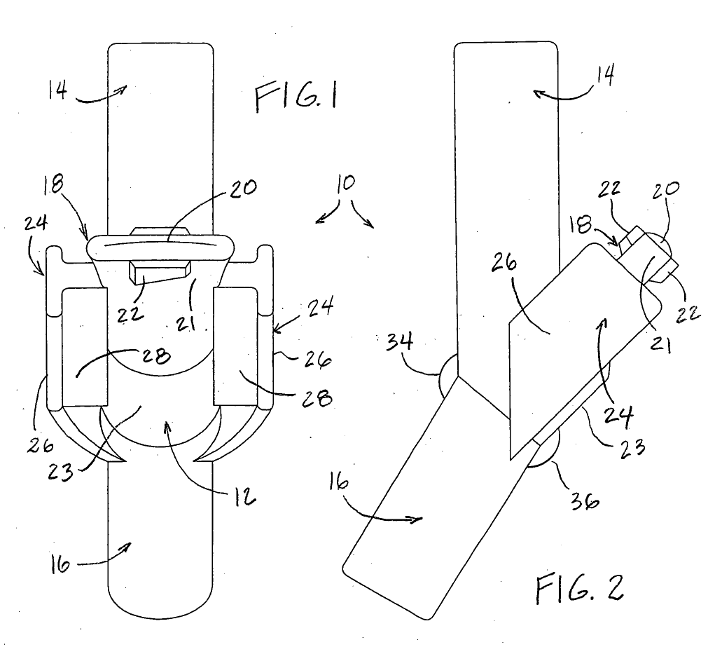

Referring now to FIG. 2, a semi-schematic side view of the valve 10 of FIG. 1 is shown. In one exemplary embodiment, the first port 14 and the second port 16 are positioned at an angle 34 of about 120 degrees to 160 degrees from one another with 135 degrees being more preferred. The slip port 18 is positioned about 135 degrees to about 180 degrees from the second port 16 with about 160 degrees being more preferred. However, other angular orientations between the three ports 14, 16, 18 may be incorporated without deviating from the spirit and scope of the present invention.

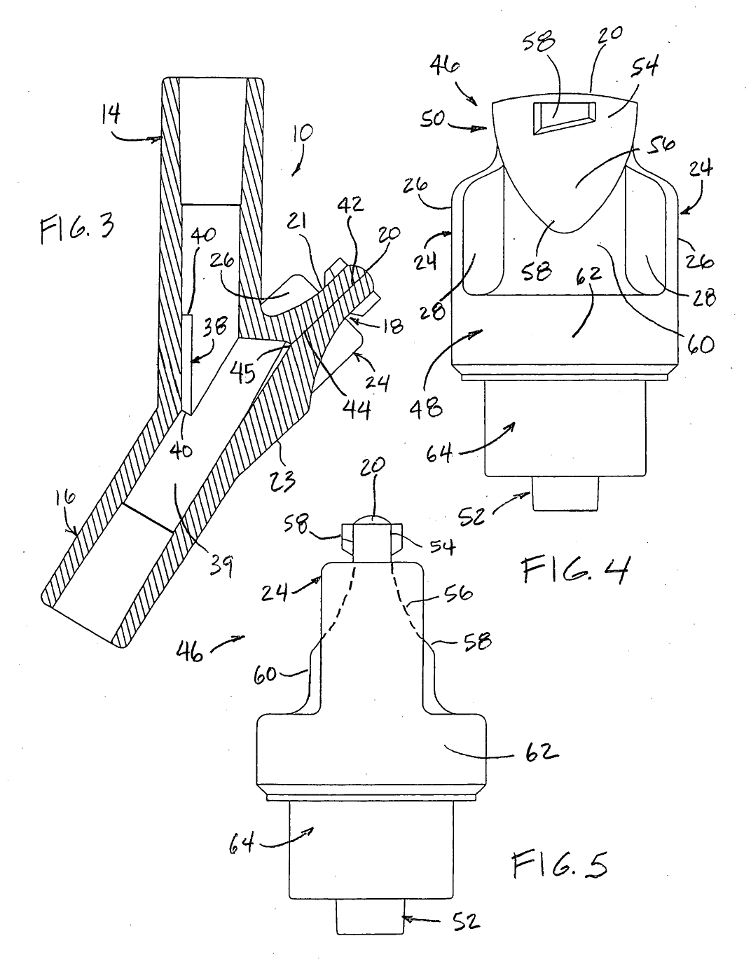

FIG. 3 is a semi-schematic cross-sectional view of the valve 10 of FIG. 1 taken from the viewing plane of FIG. 2. As shown from the cross-hatching, the valve 10 is integrally formed with the slit 20 molded or post mold cut and treated by material shrinkage or post mold mechanical set. A tubing stop 38 is integrally formed in the interior cavity 39 of the valve for delimiting movement of the tubing in both the first port 14 and the second port 16. Inserted tubes (not shown) are configured to abut the end edges 40 of the tubing stop 38 to delimit further advancement into the interior cavity 39 of the valve.

In one exemplary embodiment, the slit 20 comprises an upper slit section 42 and a lower slit section 44. Due to the geometry of the slit interiorly, i.e., the lumen, when the outer actuating pads 26 are squeezed to open the slit 20, only the upper slit section 42 opens to receive a medical implement. The lower slit section 44, and particularly the exit point 45 of the lower slit section, remains closed until further penetrated by a male tip of a medical implement. As further discussed below, the lumen is wider along the upper slit section 42 and narrows as it extends distally towards the lower slit section 44, as further discussed below. The resiliency of the slip port 18 ensures a fluid tight contact between opposing surfaces of the slit so as to prevent fluid from discharging out of the slit 20.

Referring now to FIG. 4, a semi-schematic side view of an alternative needleless injection port valve 46 provided in accordance with aspects of the present invention is shown. In one exemplary embodiment, the valve 46 comprises a valve body 48 comprising an inlet port 50 and an outlet port 52. The valve 46 is preferably formed, at least in part, from the same material or materials as the valve 10 disclosed with reference to FIG. 1. The inlet port 50 acts as a slip port and is configured to couple to a medical implement, such as a syringe, for adding or sampling fluid. The outlet port 52 is configured to couple to an IV set or a catheter assembly.

In one embodiment, the inlet port 50 comprises a self-closing opening 20 made self-closing by the resiliency of the material selected to form the valve. The self-closing opening 20 resembles a slit and the outer structure of the port defining the slit resembles a flattened open cylinder end (as best seen in FIGS. 5 and 6). The flattened upper section or upper port section 54 tapers outwardly as it extends distally towards the lower port section 56. Luer threads 58 are optionally incorporated on the inlet port 50, which are adapted to engage corresponding threads on a medical implement when the upper port section 54 is expanded by the medical implement, as further discussed below.

The convex shape bottom port section 58 of the inlet port 50 extends distally towards the main central body section 60 which then extends towards a collar 62. As further discussed below, the collar 62 overlaps a projection on the outlet assembly 64, which comprises the outlet port 52, in a tongue-and-groove configuration to couple the valve body 48 to the outlet assembly 64. A pair of levers 24 comprising actuating pads 26 and rib sections 28 are incorporated for opening the slit 20 of the inlet port 50. The pads 26 are preferably formed in a generally perpendicular configuration to the line defined by the slit.

In an exemplary embodiment, the valve body 48, which comprises the inlet port 50, the two levers 24, the central body section 60, and the collar 62, is integrally formed from an elastomer material while the outlet assembly 64 is integrally formed from a rigid plastic material, such as polycarbonate, polyurethane, or the like. More preferably, the valve body 48 is over molded to the outlet assembly 64.

FIG. 5 is a semi-schematic side view of the valve 46 of FIG. 4 viewed from a different plane. It can be seen that the flat upper port section 54 tapers outwardly towards the lower port section 56 (shown in dot-dashed lines), which extends into the central body section 60.

FIG. 6 is a semi-schematic cross-sectional side view of the valve of FIG. 5 taken from the same viewing plane. As previously discussed, in a preferred embodiment, the valve 46 is manufactured by over-molding the valve body 48 to the outlet assembly 64. The attachment is provided by forming a channel 66 between the collar 62 and the central valve body 60 over a ring-shaped projection 68 in a tongue-and-groove configuration. Interiorly, a cavity or lumen 70 defined by the interior surface of the outlet port 72 and the valve body 48 is provided for fluid flow from the inlet port 50 towards the outlet port 52, or vice versa. The flow between the two ports is regulated by the configuration of the slit 20, and more specifically by the configuration of the upper slit section 42 and the lower slit section 44. As shown, the upper and lower slit sections 42, 44 are in a closed configuration, which would prevent fluid from flowing between the two ports.

The outlet assembly 64 is shown comprising a luer threaded collar 76 and the outlet port 52. However, the outlet assembly 64 may be practiced without the threaded collar 76, which makes it a luer slip.

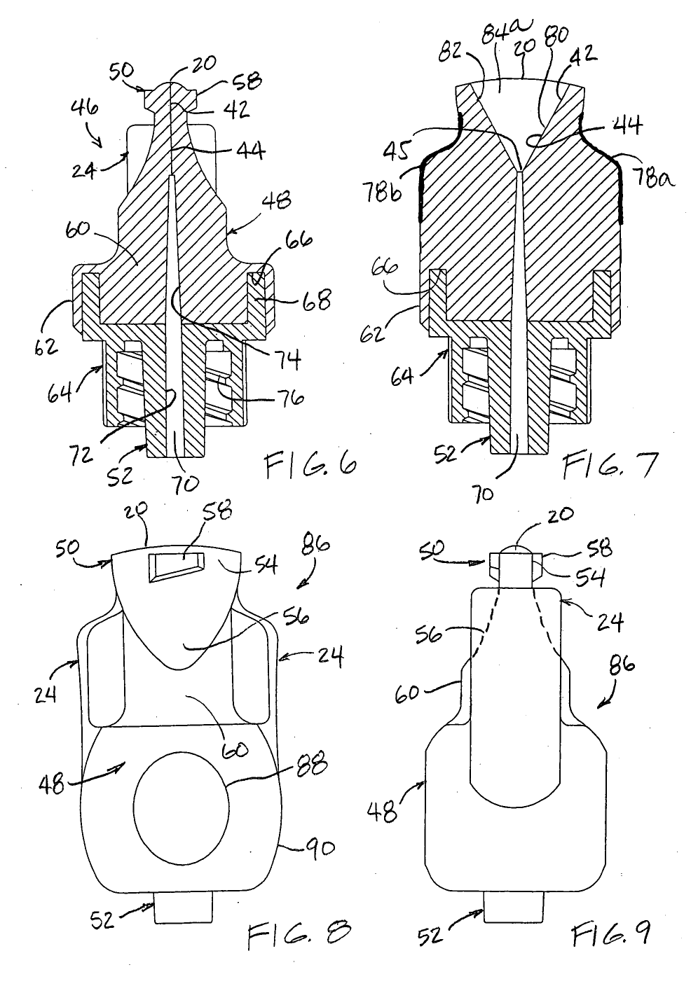

FIG. 7 is a semi-schematic cross-sectional side view of the valve 46 of FIG. 4 taken from the same viewing plane. The opening 20 comprises a funnel shape lumen having an upper section 42, a lower section 44, and an apex at the exit point 45. When the slit 20 is closed (FIG. 6), the funnel shape lumen has a generally triangular 2-dimensional configuration (FIG. 7). In other words, the lumen has three distinct sides. However, when the two levers 24 are squeezed, a force is distributed at least along the two darkened surface areas 78a, 78b adjacent the levers (shown darkened for discussion purposes only) and forces the two end edges 80, 82 of the funnel shape lumen to move toward one another. This movement causes the side walls 84a, 84b (the latter not shown) of the funnel shape lumen tangential to the slit 20 to fold and transform the 2-D funnel into a 3-D funnel as the slit 20 is opened. Because the exit point 45 is relatively narrow and the walls perpendicular to the two end edges 80, 82 at the apex small, the applied forces on the levers 24 should not open the funnel shape lumen at the exit point 45. As is readily apparent to a person of ordinary skill in the art, this configuration allows the slip port to be partially opened for receiving a medical implement while preventing fluid from discharging out the opening 20.

After opening the slit, a medical implement (not shown) may then be inserted through the opening 20 to access the interior cavity 70 of the valve 46 and be in fluid communication with the outlet port 52. When inserting the medical implement, the male tip of the medical implement abuts the exit point 45 of the lumen and mechanically opens the exit point. Fluid may then be delivered into the interior cavity 70 of the valve or be withdrawn from the interior cavity of the valve by the medical implement.

When a medical implement is connected to the inlet port 50, the interior fluid space increases from the space defined by the interior cavity 70 of the valve to include the fluid space defined by the medical implement. However, when the medical implement is removed, the fluid space reverses and decreases in size. This change in fluid space causes the valve 46 to flush out access fluid inside the interior cavity and operates as a self-flushing or positive displacement valve.

FIG. 8 is a semi-schematic side view of an alternative needleless injection port valve 86 provided in accordance with aspects of the present invention. Like the valve 46 of FIG. 4, the present valve 86 comprises an inlet port 50, an outlet port 52, and a valve body 48 comprising a pair of push levers 24. In one exemplary embodiment, various curves and contours 88, 90 are incorporated for aesthetic appeal. For example, a pair of central gripping dimples 88 may be incorporated for aesthetic appeal. In this instance, the gripping dimples 88 also provide a functional use.

FIG. 9 is a semi-schematic side view of the valve 86 of FIG. 8 from a different viewing plane. Similar to the valves discussed above, the inlet port 50 comprises a flattened upper port section 54 and a lower taper port section 56. The flattened area is configured to expand when the levers 24 are squeezed and is configured to further open when a tip of a medical implement is inserted inside the funnel shape lumen.

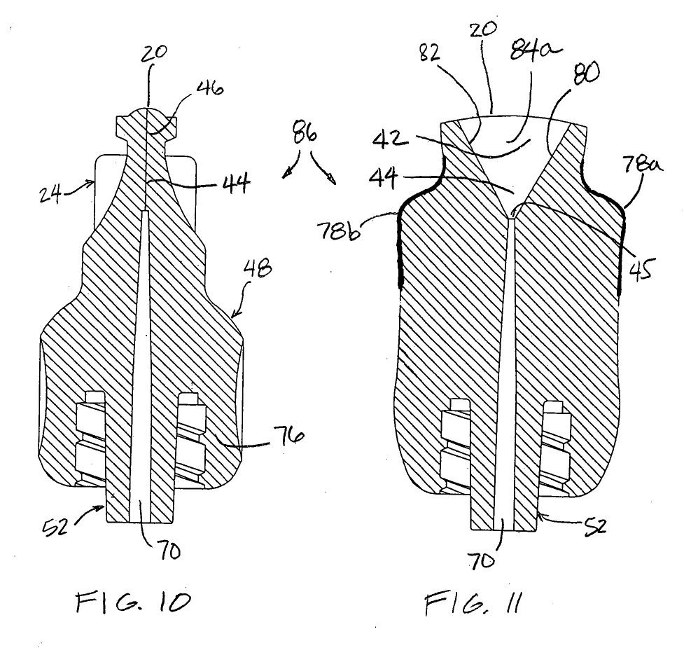

FIG. 10 is a semi-schematic cross-sectional side view of the valve 86 of FIG. 9 taken from the same viewing plane. In one exemplary embodiment, the valve 86 is a unitary construction design in which the inlet port 50, outlet port 52, and valve body 48 are integrally molded from an elastomeric material. As previously discussed, the slit 20 may be molded or post mold cut and treated either by material shrinkage or post mold mechanical set. The slit 20 inherently biases close and is open-able only if forced by prying the slit open or squeezing the levers. As with the valve of FIG. 4, the present valve is a positive displacement valve.

FIG. 11 is a semi-schematic cross-sectional side view of the valve of FIG. 8 taken from the same viewing plane. The opening 20 comprises a funnel shape lumen having an upper section 42, a lower section 44, and an apex at the exit point 45. In a closed position (See, e.g., FIG. 10), the funnel shape lumen has a generally triangular 2-dimensional configuration. When pressure is applied on the levers 24 to squeeze the levers together, a force is distributed at least along the two darkened surface areas 78a, 78b adjacent the levers (shown for discussion purposes only). The force causes the two edges 80, 82 to merge closer together while concurrently causes the two side walls 84a, 84b (the latter not shown) to flex. The slit 20 opens as a result of the flexing side walls 84a, 84b and transforms into a 3-D funnel shape lumen. A medical implement may then be inserted into the opening 20 to access the interior cavity 70 of the valve. Upon removal of the medical implement, the opening 20 self-closes and fluid inside the interior cavity 70 of the valve is displaced out of the exit opening of the outlet port 52, similar to the valve 46 described above with reference to FIG. 4.

Although limited embodiments of the needleless access valve assemblies and their components have been specifically described and illustrated herein, many modifications and variations will be apparent to those skilled in the art. For example, the various valves may incorporate luer-slips rather than luer threads, the material selected could be opaque or semi-opaque, different colors may be used, the dimensions can vary, etc. Furthermore, it is understood and contemplated that features specifically discussed for one valve embodiment may be adopted for inclusion with another valve embodiment, provided the functions are compatible. For example, certain curvatures and contours incorporated in one valve may be incorporated in another valve for aesthetic appeal and improved functionality, such as for improved gripping purposes; and rather than making an integrally molded valve, an over-molded design in which an elastomer material and a hard plastic material may be used. Accordingly, it is to be understood that the valve assemblies and their components constructed according to principles of this invention may be embodied other than as specifically described herein. The invention is also defined in the following claims.

What is claimed is:

1. A needleless injection port valve comprising a valve body, a first port comprising an exterior surface and an interior surface defining a lumen, and a second port;
the first port comprising a first configuration in which a first section of the interior surface contacts a second section of the interior surface, and a first section of the exterior surface adjacent the first section of the interior surface is spaced apart from a second section of the exterior surface adjacent the second section of the interior surface by a first distance; and
the first port comprising a second configuration in which the first and second sections of the interior surface are made to be spaced apart from one another by at least one of a medical implement and a force acting on the exterior surface of the first port, and the first section and second section of the exterior surface are spaced apart from one another by a second distance, which is larger than the first distance.

2. The needleless injection port valve of claim 1, further comprising a third port.

3. The needleless injection port valve of claim 1, further comprising at least two threaded sections positioned on the exterior surface of the first port.

4. The needleless injection port valve of claim 1, further comprising at least one rib extending from the exterior surface of the first port.

5. The needleless injection port valve of claim 4, further comprising an actuating pad extending from the at least one rib.

6. The needleless injection port valve of claim 1, wherein the lumen comprises a generally triangular shape lumen when the first port is in the first configuration.

7. The needleless injection port valve of claim 1, further comprising a third port, wherein the third port and the second port are positioned at an angle from one another.

8. A needleless injection port valve comprising a valve body, a first port comprising an exterior surface and an interior surface defining a lumen, and a second port; wherein the lumen has a shape of a funnel having a first length measured along a first plane and a second length measured along a second plane; and wherein the first length decreases and the second length increases when the first port moves from a closed position to an open position.

9. The needleless injection port valve of claim 8, further comprising a third port.

10. The needleless injection port valve of claim 8, further comprising at least two threaded sections positioned on the exterior surface of the first port.

11. The needleless injection port valve of claim 8, further comprising at least one rib extending from the exterior surface of the first port.

12. The needleless injection port valve of claim 11, further comprising an actuating pad extending from the at least one rib.

13. The needleless injection port valve of claim 8, wherein the second port comprises a threaded shroud.

14. The needleless injection port valve of claim 8, further comprising a third port, wherein the first, second, and third ports are integrally formed with the valve body.

15. A needleless injection port valve comprising a valve body, a first port comprising an exterior surface and an interior surface defining a lumen, and a second port; wherein at least two threaded sections are located on the exterior surface of the first port, and wherein the two threaded sections move away from one another when the first port moves from a closed position to an open position.

16. The needleless injection port valve of claim 15, further comprising a third port.

17. The needleless injection port valve of claim 15, wherein the second port comprises a threaded shroud.

18. The needleless injection port valve of claim 15, further comprising at least one rib extending from the exterior surface of the first port.

19. The needleless injection port valve of claim 18, further comprising an actuating pad extending from the at least one rib.

20. The needleless injection port valve of claim 15, further comprising a third port, wherein the first, second, and third ports are integrally formed with the valve body.

21. The needleless injection port valve of claim 15, wherein the valve body comprises a first body section attached to a lower body section.

22. The needleless injection port valve of claim 15, wherein the valve is made from a thermoplastic elastomer material.
